(19) United States
(12) Patent Application Publication
(10) Pub. No.: US 2015/0150491 A1
(43) Pub. Date: Jun. 4, 2015

(54) MOVEMENT ESTIMATION DEVICE, AND ACTIVITY TRACKER

(71) Applicant: Kabushiki Kaisha Toshiba, Tokyo (JP)

(72) Inventors: Kumiko Yamato, Kawasaki Kanagawa (JP); Kazunori Hashimoto, Konosu Saitama (JP); Akira Iguchi, Yokohama Kanagawa (JP)

(21) Appl. No.: 14/483,950

(22) Filed: Sep. 11, 2014

(30) Foreign Application Priority Data
Dec. 2, 2013 (JP) 2013-249690

(51) Int. Cl. A61B 5/11; G01L 7/18; A61B 5/00; G01P 15/00; G01P 13/00

(52) U.S. Cl. CPC A61B 5/1123; G01P 15/00; G01P 13/00; A61B 5/6824; A61B 5/7475; A61B 5/1118; G01L 7/18

(57) ABSTRACT

A movement estimation device according to an embodiment includes an acceleration detection unit, an acceleration intensity detection unit, a direction change detection unit, and a determination unit. The acceleration detection unit detects acceleration based on an output from an acceleration sensor configured to detect acceleration in two or more axis directions, and outputs an acceleration signal. The acceleration intensity detection unit detects an intensity of acceleration from the acceleration signal. The direction change detection unit detects a change in each axis direction of the acceleration sensor with respect to a direction of gravitational acceleration by a band-pass filter that passes a predetermined frequency range. The determination unit determines walking or a movement other than walking based on the change in each axis direction of the acceleration sensor with respect to a gravitational direction, and estimates a type of the movement other than walking based on the intensity of the acceleration.

### CROSS-REFERENCE TO RELATED APPLICATION

This application is based upon and claims the benefit of priority from the prior Japanese Patent Application No. 2013-249690 filed on Dec. 2, 2013; the entire contents of which are incorporated herein by reference.

### FIELD

An embodiment described herein relates generally to a movement estimation device, and an activity tracker.

### BACKGROUND

Conventionally, movement estimation devices having the function of a pedometer are widespread. Generally, a movement estimation device is worn on the waist or the like of a user, and measures, i.e. counts, the number of steps from the motion of the user while walking, and calculates the amount of calorie consumption and the like from the counted number of steps. A movement estimation device that uses an acceleration sensor may measure, i.e. count, the number of steps by detecting a change in the acceleration accompanying a walking movement.

When worn on the arm of a user, for example, this movement estimation device cannot distinguish between a change in the acceleration caused by walking and a change in the acceleration caused by other than walking, such as doing gymnastics, doing a clerical work, or taking a meal. That is, when this movement estimation device is worn on the arm of the user, the count of the number of steps is increased even when the user is sitting at the desk and doing a clerical work. Accordingly, the conventional movement estimation device cannot accurately count the number of steps when worn on the arm of the user, and there is a problem that an error in the amount of calorie consumption which is calculated based on the number of steps is great.

Accordingly, the conventional movement estimation device determines walking and other than walking by observing the transition of the acceleration value acquired by the acceleration sensor, and separately calculates the amount of calorie consumption for walking and other than walking, to thereby calculate the amount of calorie consumption more accurately.

However, the conventional movement estimation device cannot estimate the types of movements other than walking, and there is a problem that an accurate amount of calorie consumption cannot be calculated.

### BRIEF DESCRIPTION OF THE DRAWINGS

FIG. 1 is a block diagram showing a configuration of a movement estimation device according to a present embodiment;

FIG. 2 is a block diagram for describing a detailed circuit configuration of a gravity detection unit 15;

FIG. 3 is a diagram showing a filter property of a band-pass filter 41a;

FIG. 4A is a diagram for describing a change in the angle between the direction of gravitational acceleration and the X-axis direction of an acceleration sensor 11;

FIG. 4B is a diagram for describing a change in the angle between the direction of gravitational acceleration and the X-axis direction of the acceleration sensor 11;

FIG. 5A is a diagram for describing an example of an output from the acceleration sensor 11 during walking;

FIG. 5B is a diagram for describing an example of an output from the gravity detection unit 15 during walking;

FIG. 6A is a diagram for describing an example of an output from the acceleration sensor 11 during gymnastics;

FIG. 6B is a diagram for describing an example of an output from the gravity detection unit 15 during gymnastics;

FIG. 7A is a diagram for describing an example of an output from the acceleration sensor 11 during bending and stretching exercises;

FIG. 7B is a diagram for describing an example of an output from the gravity detection unit 15 during bending and stretching exercises;

FIG. 7C is a diagram for describing an example of an output from an acceleration energy detection unit 14 during bending and stretching exercises;

FIG. 8A is a diagram for describing an example of an output from the acceleration sensor 11 during practice swings of tennis racket;

FIG. 8B is a diagram for describing an example of an output from the gravity detection unit 15 during practice swings of tennis racket; and

FIG. 8C is a diagram for describing an example of an output from the acceleration energy detection unit 14 during practice swings of tennis racket.

### DETAILED DESCRIPTION

A movement estimation device according to an embodiment includes an acceleration detection unit, an acceleration intensity detection unit, a direction change detection unit, and a determination unit. The acceleration detection unit detects acceleration based on an output from an acceleration sensor configured to detect acceleration in two or more axis directions, and outputs an acceleration signal. The acceleration intensity detection unit detects an intensity of acceleration based on the acceleration signal from the acceleration detection unit. The direction change detection unit detects a change in each axis direction of the acceleration sensor with respect to a direction of gravitational acceleration by a band-pass filter that passes a predetermined frequency range. The determination unit determines walking or a movement other than walking based on the change in each axis direction of the acceleration sensor with respect to a gravitational direction, and estimates a type of the movement other than walking based on the intensity of the acceleration.

In the following, an embodiment will be described with reference to the drawings.

### Configuration

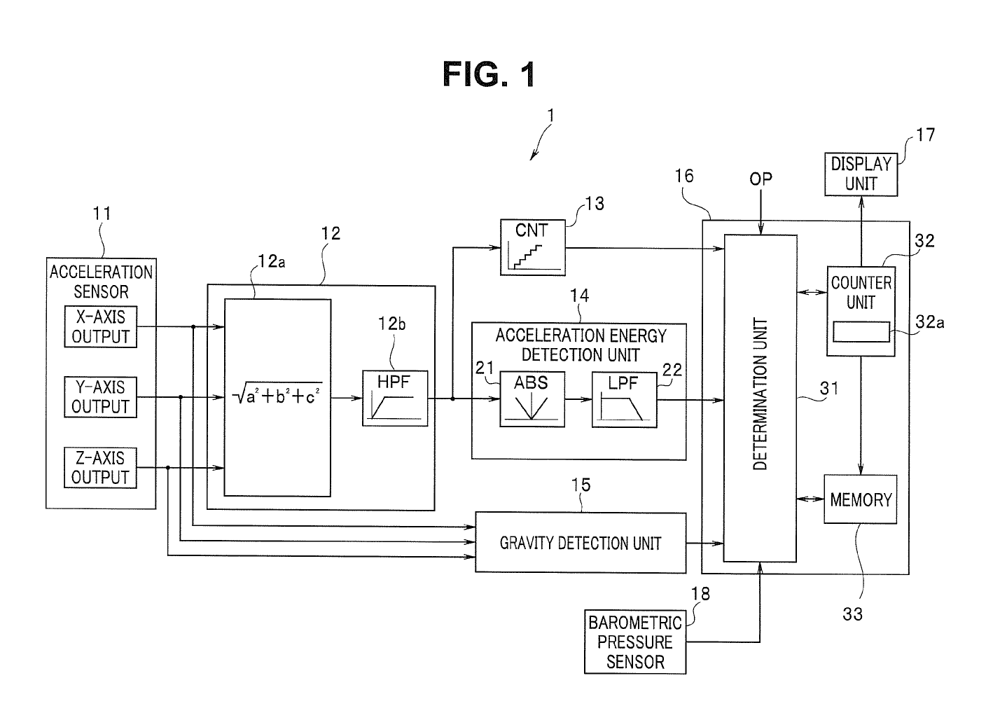

FIG. 1 is a block diagram showing a configuration of the movement estimation device according to the present embodiment. The movement estimation device described below is a wristband movement estimation device that is attached to a wristband to be wrapped around the arm of a user, for example.

A movement estimation device 1 is configured by including an acceleration sensor 11, an acceleration detection unit 12, a step counter 13, an acceleration energy detection unit 14, a gravity detection unit 15, a control unit 16, a display unit 17, and a barometric pressure sensor 18. For example, structural components other than the acceleration sensor 11, the display unit 17, and the barometric pressure sensor 18, that is, the acceleration detection unit 12, the step counter 13, the acceleration energy detection unit 14, the gravity detection unit 15, and the control unit 16 are formed in a one-chip semiconductor device as a semiconductor integrated circuit. The acceleration energy detection unit 14 includes an absolute value circuit 21, and a low-pass filter 22. Also, the control unit 16 includes a determination unit 31, a counter unit 32, and a memory 33.

Note that an on/off button for turning on or off the power of the movement estimation device 1, a reset button for resetting the count value and the like are omitted from FIG. 1, but operation signals OP from these buttons are to be input to the control unit 16.

The counter unit 32 includes a walking counter 32a. The walking counter 32a counts up the number of steps of a user during walking.

The display unit 17 is a display device such as a liquid crystal display for displaying the value of the walking counter 32a, that is, the number of steps. Also, the memory 33 stores the value of the walking counter 32a, that is, the number of steps. Furthermore, the memory 33 stores a movement estimation result from the determination unit 31.

The acceleration sensor 11 is a 3-axis acceleration sensor that includes three sensors so as to be able to detect acceleration in each of the directions along three axes (X-axis, Y-axis, Z-axis) that are orthogonal to one another, and that outputs an X-axis output, a Y-axis output, and a Z-axis output as the acceleration signals for respective axes. Each output from the acceleration sensor 11 is input to the acceleration detection unit 12 and the gravity detection unit 15.

The acceleration detection unit 12 includes a root-sum-square calculation unit 12a, and a high-pass filter (HPF) 12b.

The root-sum-square calculation unit 12a is a circuit for generating a signal of the square root of the sum of the squares of each output from the acceleration sensor 11. Here, acceleration in a plurality of directions (three in this case) is used, and the root-sum-square calculation unit 12a that generates a signal of the square root of the sum of the squares of each output is used, but a square sum calculation circuit that generates a signal of the sum of squares may also be used instead of the root-sum-square calculation unit 12a.

The high-pass filter 12b is an offset cancellation circuit for removing gravitational acceleration from the output from the root-sum-square calculation unit 12a.

Note that the acceleration sensor 11 in this case is a 3-axis acceleration sensor, but it may be any sensor that detects acceleration in two or more directions, and may be an acceleration sensor with two or more axes.

Accordingly, the acceleration detection unit 12 detects acceleration based on the output from the acceleration sensor 11 that detects acceleration in two or more directions, and outputs an acceleration signal. The acceleration signal output from the acceleration detection unit 12 is input to the step counter (CNT) 13, and the acceleration energy detection unit 14.

The step counter 13 is a counter for counting the steps based on the acceleration signal output from the acceleration detection unit 12. The step counter 13 is a counter that performs incrementation by one when there is an input at or above a predetermined threshold. The step counter 13 counts the steps based on the acceleration signal in a predetermined time period (for example, 5 seconds), and holds the count value.

The acceleration signal input to the acceleration energy detection unit 14 is input to the absolute value circuit 21. The absolute value circuit 21 calculates the absolute value of the input acceleration signal, and outputs the value to the low-pass filter 22.

The low-pass filter 22 averages the outputs from the absolute value circuit 21 and detects the intensity of the acceleration, and outputs the intensity to the determination unit 31 of the control unit 16. In this manner, the acceleration energy detection unit 14 configures the acceleration intensity detection unit that detects the intensity of the acceleration based on the acceleration signal from the acceleration detection unit 12.

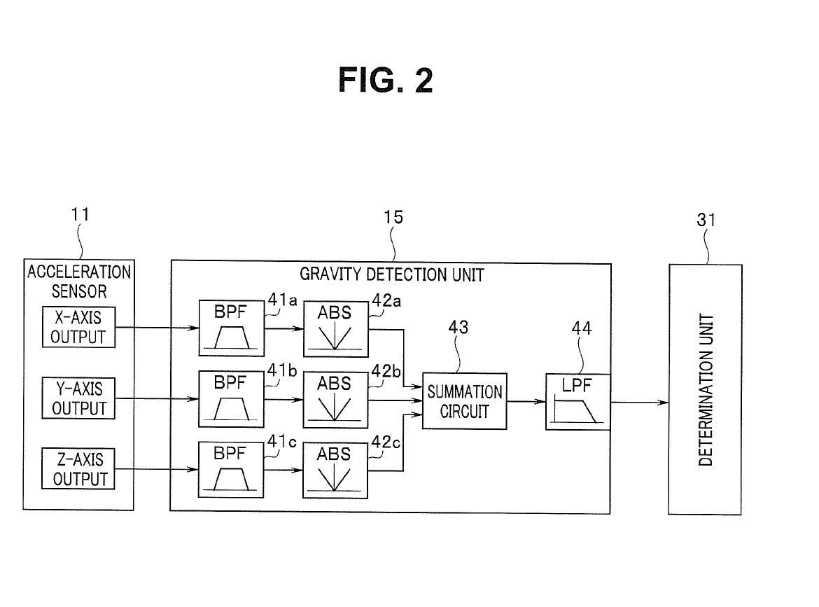

The gravity detection unit 15 detects a change in each axis direction of the acceleration sensor 11 with respect to the gravitational direction, and outputs the detection result (a determination signal described later) to the determination unit 31. Here, a detailed circuit configuration of the gravity detection unit 15 will be described with reference to FIG. 2.

FIG. 2 is a block diagram for describing a detailed circuit configuration of the gravity detection unit 15. As shown in FIG. 2, the gravity detection unit 15 is configured by including three band-pass filters 41a, 41b, and 41c, three absolute value circuits 42a, 42b, and 42c, a summation circuit 43, and a low-pass filter 44.

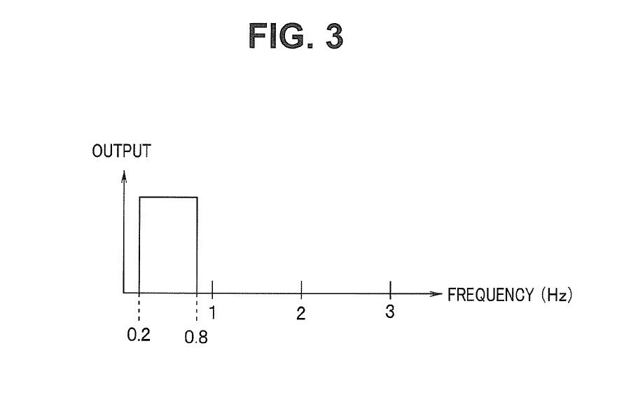

FIG. 3 is a diagram showing a filter property of the band-pass filter 41a. In FIG. 3, the horizontal axis is the frequency, and the vertical axis is the output of an output signal. Note that the filter properties of the band-pass filters 41b and 41c are the same as the filter property of the band-pass filter 41a.

Generally, the frequency of arm swing at the time of walking is about 1 Hz, and the frequency of arm swing at the time of running is about two times or more of the frequency at the time of walking. In contrast, a change in each axis direction of the acceleration sensor 11 is relatively slow and great at the time of a movement other than walking, such as doing gymnastics or taking a meal.

As shown in FIG. 3, each of the band-pass filters 41a to 41c is a filter for passing frequency components between 0.2 Hz and 0.8 Hz. By using the band-pass filters 41a to 41c having the filter property shown in FIG. 3, signals according to a change are output from the band-pass filters 41a to 41c only when each axis direction of the acceleration sensor 11 has changed slowly and greatly with respect to the gravitational direction. As described, the gravity detection unit 15 configures the direction change detection unit that detects a change in each axis direction of the acceleration sensor 11 with respect to the gravitational direction by the band-pass filters 41a to 41c that pass a predetermined range. More specifically, the gravity detection unit 15, which is the direction change detection unit, detects a change in the angle between the direction of gravitational acceleration and each axis direction of the acceleration sensor 11. That is, since the acceleration sensor 11 is constantly under the influence of gravity, the value of the acceleration sensor 11 also changes for each axis due to a change in the angle between the direction of gravitational acceleration and each axis direction.

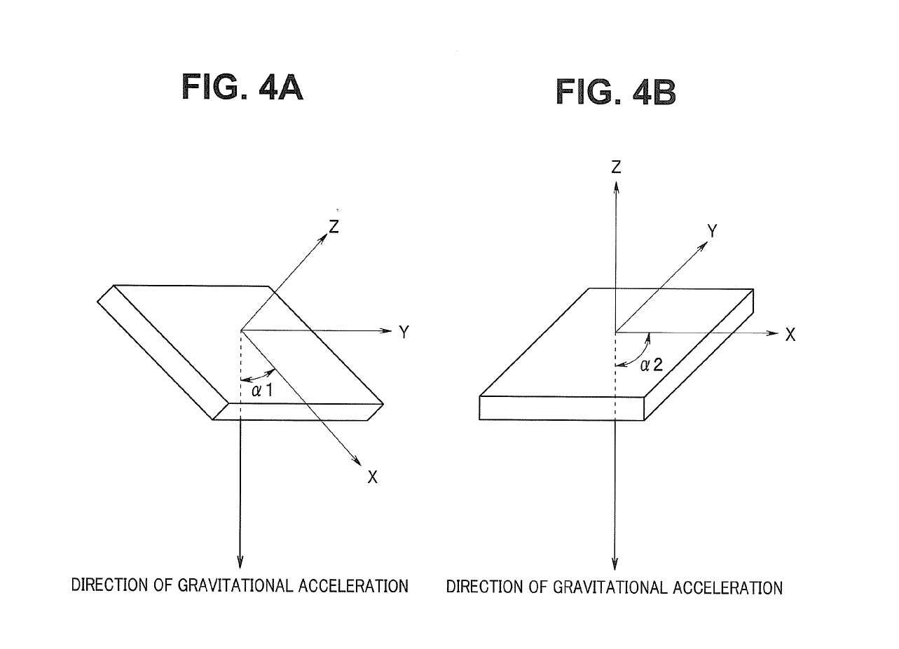

FIGS. 4A and 4B are diagrams for describing a change in the angle between the direction of gravitational acceleration and the X-axis direction of the acceleration sensor 11.

In the case where the acceleration sensor 11 is oriented as shown in FIG. 4A, the angle between the direction of gravitational acceleration and the X-axis direction of the acceleration sensor 11 is a α1. When the orientation of the acceleration sensor 11 is changed to that shown in FIG. 4B by a movement of the user, the angle between the direction of gravitational acceleration and the X-axis direction of the acceleration sensor 11 becomes α2. When the angle between the direction of gravitational acceleration and the X-axis direction of the acceleration sensor 11 changes, that is, when α1 changes to α2, the value of the X-axis output from the acceleration sensor 11 is also changed. The gravity detection unit 15 passes, by the band-pass filter 41a, the component of a change (motion) in the angle between the direction of gravitational acceleration and the X-axis direction of the acceleration sensor 11, and detects the motion. Detection is performed in the same manner with respect to the Y-axis direction and the Z-axis direction of the acceleration sensor 11, and changes in the angles between the direction of gravitational acceleration and the respective axis directions of the acceleration sensor 11 are detected.

Outputs from the band-pass filters 41a to 41c are input to the absolute value circuits 42a to 42c, respectively. The absolute value circuits 42a to 42c calculate the absolute values of the outputs from the band-pass filters 41a to 41c, respectively, and output the same to the summation circuit 43.

The summation circuit 43 adds up the outputs from the absolute value circuits 42a to 42c, and outputs the same to the low-pass filter 44. The low-pass filter 44 averages the outputs from the summation circuit 43, and generates a determination signal. This determination signal is output to the determination unit 31 of the control unit 16.

The determination unit 31 determines whether the current movement is walking/running (hereinafter, walking and running are collectively referred to as walking unless specified otherwise) or other than walking, by comparing the determination signal from the gravity detection unit 15 with a predetermined threshold.

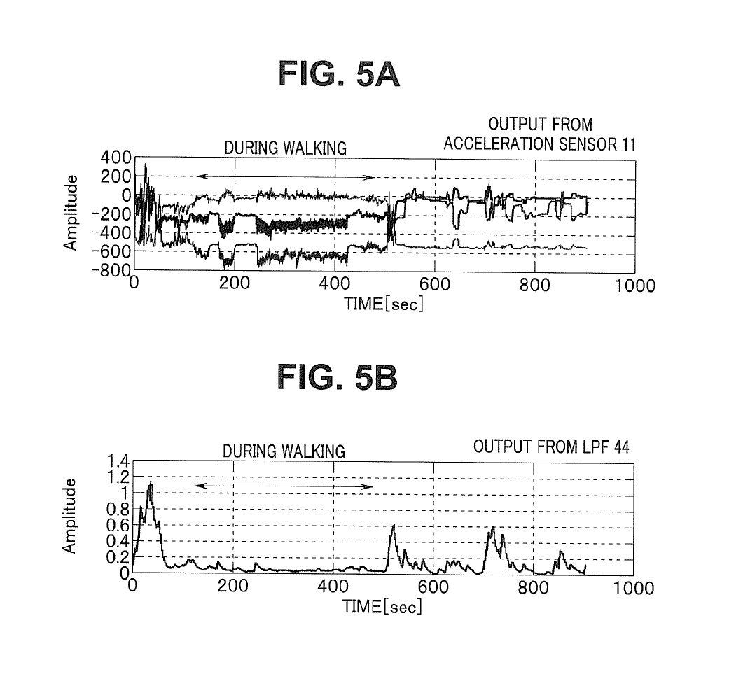

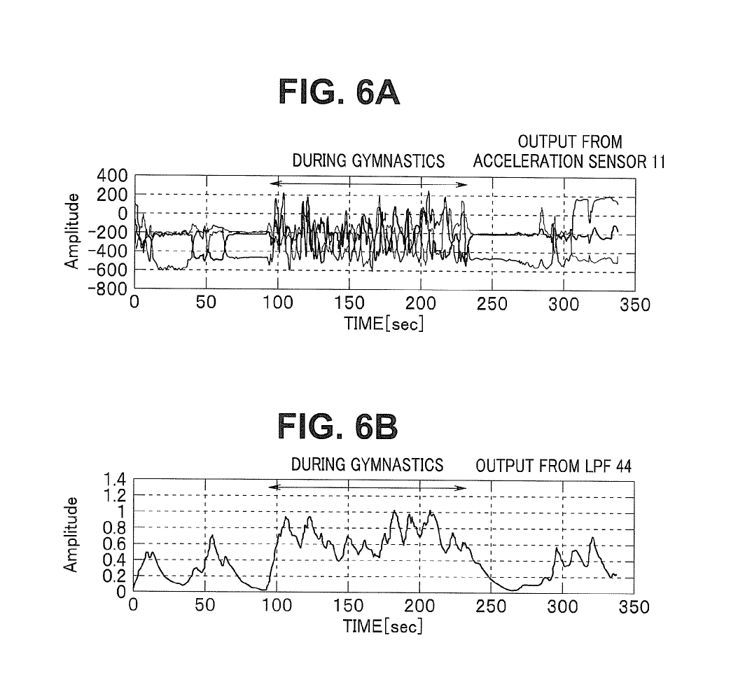

FIGS. 5A and 5B are diagrams for describing examples of outputs from the acceleration sensor 11 and the gravity detection unit 15 during walking, and FIGS. 6A and 6B are diagrams for describing examples of outputs from the acceleration sensor 11 and the gravity detection unit 15 during gymnastics. Note that FIG. 5A shows an output from the acceleration sensor 11 during walking, and FIG. 5B shows an output from the gravity detection unit 15 during walking. Similarly, FIG. 6A shows an output from the acceleration sensor 11 during gymnastics, and FIG. 6B shows an output from the gravity detection unit 15 during gymnastics.

Also, in FIGS. 5A and 5B, outputs during walking are shown from around 120 seconds to 460 seconds, and in FIGS. 6A and 6B, outputs during gymnastics are shown from around 100 seconds to 230 seconds.

As shown in FIG. 5B, during walking, the output from the gravity detection unit 15, or more specifically, the output from the low-pass filter 44, is small, almost none. In contrast, as shown in FIG. 6B, during a movement other than walking, gymnastics in this case, the output from the gravity detection unit 15, or more specifically, the output from the low-pass filter 44, is great. The determination unit 31 determines whether the current movement is walking or other than walking, based on such an output from the gravity detection unit 15.

In the case of determining that the current movement is walking based on the output from the gravity detection unit 15, the determination unit 31 adds the count value of the step counter 13 to the walking counter 32a of the counter unit 32.

Specifically, the determination unit 31 determines walking or a movement other than walking based on the output from the gravity detection unit 15 at predetermined intervals (for example, 5 seconds), and when walking is determined, the determination unit 31 adds the count value of the step counter 13 for the duration of the immediately preceding interval (for example, 5 seconds) to the count value of the walking counter 32a.

Note that when each reset button (not shown) or the like is pressed, and an operation signal OP which is a reset signal is generated, the count value of the walking counter 32a is set to zero so that the number of steps becomes zero.

As described above, the count value of the walking counter 32a, that is, the number of steps, is displayed on the display unit 17.

Also, the count value of the walking counter 32a is recorded in the memory 33 every specific period of time.

On the other hand, in the case of determining that the current movement is other than walking by comparing the determination signal from the gravity detection unit 15 with a predetermined threshold, the determination unit 31 estimates the type of the movement that is other than walking based on the intensity of acceleration from the acceleration energy detection unit 14.

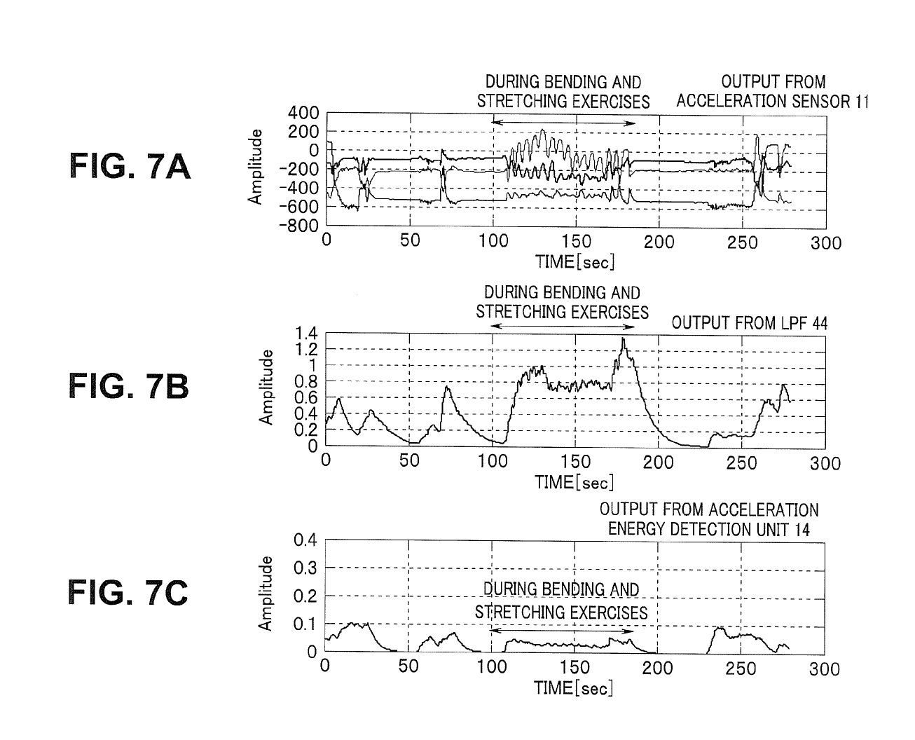

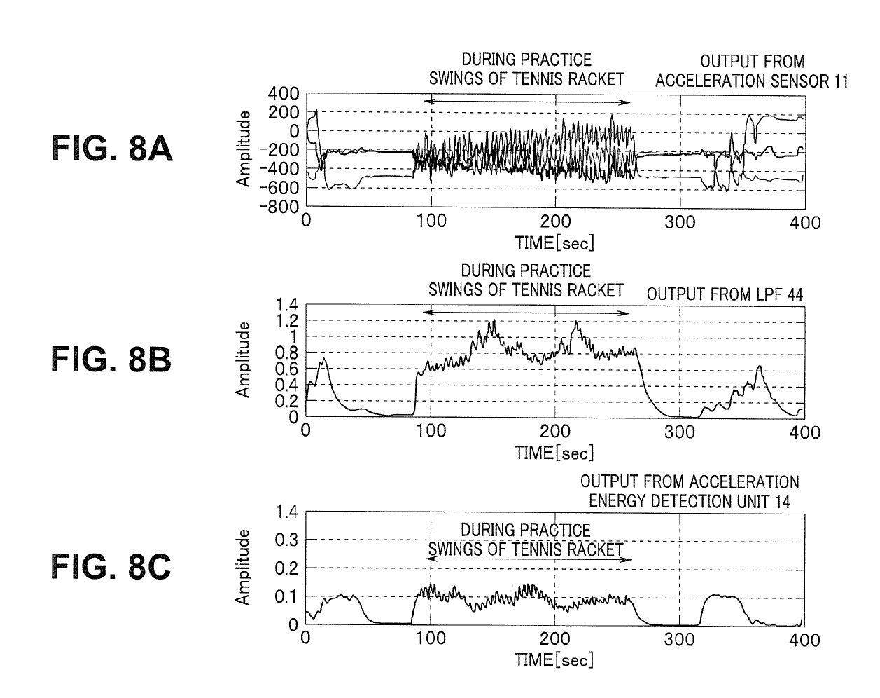

FIGS. 7A, 7B, and 7C are diagrams for describing examples of outputs from the acceleration sensor 11, the gravity detection unit 15, and the acceleration energy detection unit 14 during bending and stretching exercises, and FIGS. 8A, 8B, and 8C are diagrams for describing examples of outputs from the acceleration sensor 11, the gravity detection unit 15, and the acceleration energy detection unit 14 during practice swings of tennis racket. Note that FIG. 7A shows an output from the acceleration sensor 11 during bending and stretching exercises, FIG. 7B shows an output from the gravity detection unit 15 during bending and stretching exercises, and FIG. 7C shows an output from the acceleration energy detection unit 14 during bending and stretching exercises. Similarly, FIG. 8A shows an output from the acceleration sensor 11 during practice swings of tennis racket, FIG. 8B shows an output from the gravity detection unit 15 during practice swings of tennis racket, and FIG. 8C shows an output from the acceleration energy detection unit 14 during practice swings of tennis racket.

Also, in FIGS. 7A, 7B, and 7C, outputs during bending and stretching exercises are shown from around 100 seconds to 180 seconds, and in FIGS. 8A, 8B, and 8C, outputs during practice swings of tennis racket are shown from around 100 seconds to 260 seconds.

The determination unit 31 determines movements other than walking based on the outputs from the gravity detection unit 15 shown in FIGS. 7B and 8B, and estimates movements other than walking (bending and stretching exercises and practice swings of tennis racket) based on the outputs from the acceleration energy detection unit 14 shown in FIGS. 7C and 8C.

Note that movements other than bending and stretching exercises and practice swings of tennis racket may also be estimated based on the outputs from the acceleration energy detection unit 14. Also, the determination unit 31 may categorize movement intensities into low, medium, and high, for example, by comparing outputs from the acceleration energy detection unit 14 with a predetermined threshold, and estimate a movement of a movement intensity of any of low, medium, and high, and determine the amount of calorie consumption (energy consumption) according to each movement intensity.

Then, the determination unit 31 stores the estimation result in the memory 33. Thus, the memory 33 configures a storage unit for storing estimation results estimated by the determination unit 31.

Note that in the estimation of the type of a movement other than walking, a measurement value of the barometric pressure sensor 18 may also be used in addition to the intensity of acceleration from the acceleration energy detection unit 14. That is, in the case of determining that the current movement is other than walking, the determination unit 31 estimates the type of the movement that is other than walking based on the intensity of acceleration from the acceleration energy detection unit 14 and a measurement value from the barometric pressure sensor 18.

The barometric pressure sensor 18 measures atmospheric pressure at the time of the movement estimation device 1 being worn on the arm of the user, and outputs the measurement result to the determination unit 31. The determination unit 31 detects the height of the arm of the user wearing the movement estimation device 1 based on the measurement value from the barometric pressure sensor 18. When the user is doing a clerical work such as typing on the keyboard, the height of the arm hardly changes, and the measurement value of the barometric pressure sensor 18 does not change much. On the other hand, when the user is doing gymnastics, the height of the arm changes frequently, and the measurement value of the barometric pressure sensor 18 changes frequently. That is, by providing the barometric pressure sensor 18 to the movement estimation device 1, the position of the arm of the user may be detected. The determination unit 31 determines a movement other than walking from the output from the gravity detection unit 15, for example, and may estimate a clerical work in the case where there is no change in the output from the barometric pressure sensor 18 (in the case where there is no change in the height of the aim).

In this manner, in the case where the current movement is determined to be other than walking, the determination unit 31 may more accurately estimate the type of the movement that is other than walking by performing movement estimation based on the intensity of acceleration from the acceleration energy detection unit 14 and the measurement value of the barometric pressure sensor 18.

### Effect

When a user wears the movement estimation device 1 on the arm and makes a movement, the output value for each axis detected by the acceleration sensor 11 is input to the acceleration detection unit 12 and the gravity detection unit 15. The output value for each axis detected by the acceleration sensor 11 is input to the acceleration detection unit 12, and an acceleration signal is detected. The acceleration signal detected by the acceleration detection unit 12 is input to the acceleration energy detection unit 14, and the intensity of acceleration is detected. This intensity of acceleration is input to the determination unit 31.

Also, the output values for respective axes detected by the acceleration sensor 11 are input to the band-pass filters 41a to 41c of the gravity detection unit 15. The frequency component for walking is removed by the band-pass filters 41a to 41c for passing the frequency components between 0.2 Hz and 0.8 Hz, and a signal according to a change is output only when each axis direction of the acceleration sensor 11 has changed slowly and greatly with respect to the gravitational direction.

Absolute values of the outputs from the band-pass filters 41a to 41c are calculated by the absolute value circuits 42a to 42c, respectively. Then, the outputs from the absolute value circuits 42a to 42c are added at the summation circuit 43 and then averaged by the low-pass filter 44, and output to the determination unit 31 as a determination signal.

When a user is making a movement other than walking, each axis direction of the acceleration sensor 11 changes slowly with respect to the gravitational direction. The band-pass filters 41a to 41c output signals according to a change only when each axis direction of the acceleration sensor 11 changes slowly and greatly with respect to the gravitational direction. Accordingly, the determination unit 31 may determine whether the current movement is walking or other than walking by comparing the determination signal generated based on the outputs from the band-pass filters 41a to 41c with a predetermined threshold.

In the case of determining that the current movement is other than walking, the determination unit 31 estimates the type of the movement that is other than walking based on the intensity of acceleration from the acceleration energy detection unit 14. Note that the determination unit 31 may more accurately estimate the type of the movement that is other than walking by detecting the height of the arm of the user from the measurement value of the barometric pressure sensor 18, in addition to using the intensity of acceleration from the acceleration energy detection unit 14.

The determination unit 31 stores the estimation result of the movement of the user in the memory 33. Here, at the time of storing the estimation result of the movement of the user in the memory 33, the determination unit 31 stores the estimation result together with the current time. By storing the estimation result of the movement of the user and the current time in association with each other, the movement estimation device 1 may acquire the estimation result of the movement of the user for one day.

By acquiring the estimation result of the movement of a user for one day, the movement estimation device 1 may acquire the movement pattern of the user for one day and the lifestyle such as how many hours gymnastics were done and at what time a meal was taken. For example, by mounting the movement estimation device 1 on the arm of a patient afflicted with a lifestyle-related disease, and acquiring the movement pattern of the patient for one day, a doctor is enabled to give appropriate advices to the patient for improving the living environment and the lifestyle habits.

In this manner, according to the movement estimation device 1 of the embodiment described above, the types of movements other than walking may be estimated.

Note that the determination unit 31 may also calculate the amount of calorie consumption according to the current movement. For example, the determination unit 31 calculates the amount of calorie consumption according to walking or the type of a movement that is other than walking based on the intensity of acceleration from the acceleration energy detection unit 14. Alternatively, in the case where the current movement is determined to be walking, the determination unit 31 calculates the amount of calorie consumption by a calculation formula according to walking, and in the case where the current movement is determined to be a movement other than walking, the determination unit 31 calculates the amount of calorie consumption by a calculation formula according to the type of the movement that is other than walking. The movement estimation device 1 may thereby accurately calculate the amount of calorie consumption for one day of the user.

The movement estimation device described above is a wristband movement estimation device that is attached to a wristband to be wrapped around the arm of a user, but it may also take another form. For example, the movement estimation device described above may be a wristwatch equipped with a movement estimation function embedded therein. Moreover, the movement estimation device may be provided with a wireless or wired communication interface, and may be enabled to output a movement estimation result to a smartphone, a personal computer, and the like.

Note that, in the present embodiment, the movement estimation device is described, but an activity tracker provided with the function of the movement estimation device described above may alternatively be used instead of the movement estimation device, for example.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A movement estimation device comprising:
an acceleration detection unit configured to detect acceleration based on an output from an acceleration sensor configured to detect acceleration in two or more axis directions, and to output an acceleration signal;
an acceleration intensity detection unit configured to detect an intensity of acceleration based on the acceleration signal from the acceleration detection unit;
a direction change detection unit configured to detect a change in each axis direction of the acceleration sensor with respect to a direction of gravitational acceleration by a band-pass filter that passes a predetermined frequency range; and
a determination unit configured to determine walking or a movement other than walking based on an output from the direction change detection unit, and to estimate a type of the movement other than walking based on the intensity of the acceleration.

2. The movement estimation device according to claim 1,
wherein the movement estimation device is configured to be worn on an arm,
a barometric pressure sensor configured to detect atmospheric pressure at a height of the arm on which the movement estimation device is worn is included, and
the determination unit estimates the type of the movement other than walking based on a measurement value detected by the barometric pressure sensor.

3. The movement estimation device according to claim 1, wherein the acceleration sensor is a 3-axis acceleration sensor capable of detecting acceleration in directions along three axes that are orthogonal to one another.

4. The movement estimation device according to claim 1, further comprising:
a storage unit configured to store an estimation result estimated by the determination unit.

5. The movement estimation device according to claim 4, wherein the storage unit stores the estimation result estimated by the determination unit together with a current time.

6. The movement estimation device according to claim 1, wherein the band-pass filter is a filter that is configured to pass frequency components between 0.2 Hz and 0.8 Hz.

7. The movement estimation device according to claim 1, wherein the determination unit determines the walking or the movement other than walking by comparing an output from the direction change detection unit with a predetermined threshold.

8. The movement estimation device according to claim 1, wherein the determination unit calculates an amount of calorie consumption according to the walking or the type of the movement other than walking.

9. The movement estimation device according to claim 1, wherein the determination unit estimates movement intensity by comparing an output from the acceleration intensity detection unit with a predetermined threshold, and calculates an amount of calorie consumption according to the movement intensity.

10. The movement estimation device according to claim 1, further comprising:
a wireless or wired communication interface,
wherein the communication interface is configured to output an estimation result estimated by the determination unit to an external device.

11. An activity tracker comprising:
a configuration of the movement estimation device according to claim 1.

12. The activity tracker according to claim 11,
wherein the activity tracker is configured to be worn on an arm,
a barometric pressure sensor configured to detect atmospheric pressure at a height of the arm on which the activity tracker is worn is included, and
the determination unit estimates the type of the movement other than walking based on a measurement value detected by the barometric pressure sensor.

13. The activity tracker according to claim 11, wherein the acceleration sensor is a 3-axis acceleration sensor capable of detecting acceleration in directions along three axes that are orthogonal to one another.

14. The activity tracker according to claim 11, further comprising:
a storage unit configured to store an estimation result estimated by the determination unit.

15. The activity tracker according to claim 14, wherein the storage unit stores the estimation result estimated by the determination unit together with a current time.

16. The activity tracker according to claim 11, wherein the band-pass filter is a filter that is configured to pass frequency components between 0.2 Hz and 0.8 Hz.

17. The activity tracker according to claim 11, wherein the determination unit determines the walking or the movement other than walking by comparing an output from the direction change detection unit with a predetermined threshold.

18. The activity tracker according to claim 11, wherein the determination unit calculates an amount of calorie consumption according to the walking or the type of the movement other than walking.

19. The activity tracker according to claim 11, wherein the determination unit estimates movement intensity by comparing an output from the acceleration intensity detection unit with a predetermined threshold, and calculates an amount of calorie consumption according to the movement intensity.

20. The activity tracker according to claim 11, further comprising:
a wireless or wired communication interface,
wherein the communication interface is configured to output an estimation result estimated by the determination unit to an external device.
